(19) United States
(12) Patent Application Publication
(10) Pub. No.: US 2011/0081664 A1
(43) Pub. Date: Apr. 7, 2011

(54) MULTIPURPOSE MICROFLUIDIC DEVICE FOR MIMICKING A MICROENVIRONMENT WITHIN A TUMOR

(75) Inventors: Neil St. John Forbes, Belchertown, MA (US); Rachel W. Kasinskas, Amesbury, MA (US); Colin L. Walsh, San Francisco, CA (US); Brett M. Babin, Chester, NH (US); Bhushan J. Toley, Amherst, MA (US)

(73) Assignee: University of Massachusetts, Boston, MA (US)

(21) Appl. No.: 12/580,592

(22) Filed: Oct. 16, 2009

Related U.S. Application Data
(60) Provisional application No. 61/106,480, filed on Oct. 17, 2008.

(51) Int. Cl. G01N 33/53; C12M 3/00; C12M 3/06; C12N 5/071; C12Q 1/06

(52) U.S. Cl. 435/7.23; 435/289.1; 435/303.1; 435/325; 435/39; 435/7.21; 435/7.24

(57) ABSTRACT

The present application relates generally to a novel microfluidic device for the in it propagation of neoplastic cellagregates under conditions that mimic the physiological microenvironment found in tumors. The invention also describes methods of screening for therapeutic test agents and protocols that target proliferating and quiescent neoplastic cells within tumors.

### PRIORITY CLAIMS AND RELATED APPLICATIONS

This application claims the benefit of U.S. Provisional Application No. 61/106,480, filed Oct. 17, 2008, the entire content of which is expressly incorporated herein by reference.

### GOVERNMENT RIGHTS

The United States Government has certain rights to the invention pursuant to financial support from the National Institutes of Health (Grant Nos. 1R21CA112335-01A and 1R01CA120825-01A1) and the National Science Foundation (Grant No. DMI-0531171) to the University of Massachusetts.

### FIELD OF THE INVENTION

The invention generally relates to a novel microfluidic device for mimicking a microenvironment within tissues. More particularly, the invention relates to microfluidic devices that mimic the physiological microenvironment found in tumors and methods and applications of these devices in developing targeted cancer therapeutics.

### BACKGROUND OF THE INVENTION

Most chemotherapeutics are only effective against proliferating, cancer cells and have only limited efficacy on quiescent cells. In addition, poor perfusion limits the ability of systemically administered drugs from penetrating interstitial tissue in sufficient concentrations to be effective. Controlling the delivery of therapeutics to all tumor sites and targeting both proliferating and quiescent cancer cells is essential if cancer therapies are to be successful at preventing recursion and metastatic disease.

The heterogeneity of cellular microenvironments in tumors severely limits the efficacy of most cancer therapies. A major cause of this heterogeneity is the geometric arrangement of blood vessels within a tumor. Non-uniform delivery of nutrients and removal of waste products affects the proliferation of different cell-types within the tumor. Hence, next to the vessel wall, tumor tissue is well supplied with nutrients and rapidly proliferates. Further from the blood supply, the concentrations of nutrients decrease and cells become quiescent, apoptotic and eventually necrotic. Furthermore, extracellular pH decreases with increasing distance from blood vessels.

Two well-established methods of creating microenvironment gradients in vitro are tumor spheroids and sandwich cultures. Spheroids are spherical clusters of cells grown suspended in culture medium. Nutrient diffusion through concentric cell layers creates the microenvironment gradients typically observed in human tumors in vivo. However, these gradients are difficult to study because the interior regions of spheroids cannot be observed microscopically and can only be investigated by physical cell dissociation. Sandwich cultures create large millimeter-scale gradients by constraining monolayers of cells between glass slides. Sandwich cultures enable the observation of cellular microenvironments using standard fluorescence microscopy, but do not capture the cell-cell interactions or interstitial diffusion resistances present in tumors.

Previous efforts to create micron-scale cellular bioreactors can be divided into two groups: those that contain monolayers and those that contain three-dimensional tissue. The design goal for most monolayer devices was to create a homogeneous environment without microenvironment gradients. A nanoliter bioreactor and a high aspect ratio device have been designed to contain cell culture chambers molded in polydimethylsiloxane (PDMS) and microfluidic medium flow, in order to mimic the environment in cell culture flasks and microtiter plates. Micro-fabricated cell culture chips composed of polymethylmethacrylate (PMMA) with intergraded heat regulation and pH control have been shown to support cell growth for two weeks and have similar gene expression to cells grown in culture flasks. Mammary epithelial cells grown in microchannels constructed from enzymatically crosslinked gelatin exhibit morphological growth patterns similar to in vivo tumors. PDMS microchannel bioreactors have also been used to show that cells grow best with moderate medium flow and shear stress.

There is, therefore, an urgent need for novel experimental models and devices that mimic a microenvironment within tumors that can be used to discover cancer therapies that effectively target both proliferating and quiescent cancer cells.

### SUMMARY OF THE INVENTION

The invention discloses a novel Microfluidic device that mimics a physiological microenvironment found in tumors. For example, the microfluidic device may be designed to mimic a microenvironment gradient within a tumor tissue, thus providing a useful platform to accurately quantify the penetration of novel therapeutics, measure their long-term effects on tissue viability, and assess their overall efficacy.

By constraining a three-dimensional cell mass within a cell culture chamber, linear microenvironment gradients are formed that are perpendicular to the nutrient source and that predictably reproduce the diversity of cell-types and environments surrounding blood vessels in tumors. The invention provides a novel and effective experimental model to design new therapeutic strategies that specifically target the quiescent, therapeutically resistant microenvironments that are unique to tumors and not present in normal tissue.

In one aspect, the invention generally relates to a microfluidic device for mimicking a physiological characteristic of a three-dimensional cell mass. The microfluidic device includes a cell culture chamber for culturing a cell aggregate (the cell culture chamber having a proximal end and a distal end); a flow channel in fluid communication with the cell culture chamber at the proximal end; one or more inlets connected to the flow channel for medium in-flow or cell aggregate packing; one or more outlets connected to the flow channel for medium out-flow or removal of cell debris; and a flow system for maintaining medium flow. A filter is placed at the distal end of the cell culture chamber. The filter allows fluid flow-through while retains the cell aggregate in place within the cell culture chamber. At least a portion of the cell culture chamber is optically accessible through a transparent window.

In certain embodiments, the device may have a temperature-controllable housing for placing the cell culture chamber therein.

In some embodiments, the cell culture chamber has a planar transparent window allowing the cell aggregate to present a uniformed sample surface. A portion of the cell chamber can be optically accessible to fluorescence and time-lapse microscopy and the internal surface of the cell culture chamber can have a cell non-adhesive or cell-adhesive surface.

The cell aggregate may be aggregates of neoplastic cells, apoptotic, quiescent and proliferating cells, cancer stem cells or tumor spheroids.

In another aspect, the invention generally relates to a microfluidic device for mimicking, a physiological characteristic of three-dimentional cell masses. The microfluidic device includes a plurality of cell culture chambers for culturing a plurality of cell aggregates. Each cell culture chamber has a proximal end and a distal end. The microfluidic device further includes one or more flow channels in fluid communication with each cell culture chamber at the proximal end; one or more inlets connected to each flow channel for medium in-flow or cell aggregate packing; one or more outlets connected to each flow channel for medium out-flow or removal of cell debris; and a flow system for maintaining medium flow in the one or more flow channels. A filter is placed at the distal end of each cell culture chamber and allows fluid flow-through while retaining the cell aggregate in place within the cell culture chamber. At least a portion of the cell culture chamber is optically accessible through a transparent window.

In yet another aspect, the invention generally relates to a method for mimicking a physiological characteristic of a three-dimensional cell mass. The method includes: providing a cell culture chamber for holding a cell aggregate, the cell culture chamber having a proximal end and a distal end; providing a flow channel in fluid communication with the cell culture chamber at the proximal end; seeding a cell aggregate in the cell culture chamber, wherein a filter at the distal end of the cell culture chamber retains the cell aggregate in the cell culture chamber; and culturing the cell aggregate in the cell culture chamber, wherein culture medium flowing through the flow channel can diffuse into cell culture chamber. The method can mimic a characteristic of the microenvironments surrounding blood vessels in a tissue, such as a tumor tissue. The method may further include measuring a transport or targeting property of an agent in a tumor tissue. In certain embodiments, the method may further include a radiation step.

In yet another aspect, the invention generally relates to a method for screening a compound for a therapeutic property. The method includes: providing a cell culture chamber for holding a cell aggregate, the cell culture chamber having a proximal end and a distal end; providing a flow channel in fluid communication with the cell culture chamber at the proximal end; culturing the cell aggregate in the cell culture chamber, wherein a filter at the distal end of the cell culture chamber retains the cell aggregate in the cell culture chamber; adding a test agent to the culture medium flowing through the flow channel; allowing diffusion of the test agent from the flow channel to the cell culture chamber; and recording a change in a therapeutic property of the cell aggregate in response to the presence of the test agent. In certain embodiments, the method may further include a radiation step.

The test agent may be a chemotherapeutic agent, an agent that induces apoptosis of cancer stem cells in the cell aggregate, an antibody or a small molecule. The test agent may also be a cell, a genetically-engineered cell, an immune cell or an anti-cancer bacterium. In some embodiments, the test agent may be labeled.

### BRIEF DESCRIPTION OF THE DRAWINGS

FIG. 1 schematically shows an exemplary depiction of how the microenvironments created in the microfluidic device mimics those surrounding blood vessels in tumors. (A) Nutrient and waste gradients away from vessels creates regions of proliferating quiescent, and necrotic tissue. Drugs have varying penetration capabilities. Some penetrate deeply (stars), while others do not (crosses). Engineered bacteria (ovals) have the potential to penetrate to therapeutically resistant regions. (B) Linear, observable microenvironment gradients in the cell culture chamber of the microfluidic device have a similar pattern to those surrounding blood vessels in tumors and result in regions of proliferating, quiescent, and necrotic cells. (C) Depiction of the conceptual concentration profiles of nutrients, drugs, and wastes around blood vessels that are emulated by the device.

FIG. 2 schematically shows an exemplary design of the microfluidic tumor device. (A) Top-view of the device showing the arrangement of the medium/cell inlet, the packing outlet, and the medium outlet. (B) Working device with flow inlets, outlets and check valve attached. (C) Cross-section view of the device showing holes through the microscope slide used to connect to the fluid flow. (D) Expanded image of the packing chamber in the center of the device in (A). (E) Adjusted dimensions of the cell retention filter: post width, post length, and gap width.

FIG. 3 shows an exemplary tumor tissue chamber and cell-retention filter. (A) Bright field image of the chamber and filter. (B) Bright field image of tissue packed into the chamber. Scale bar is 100 mm. (C) Scanning electron microscope image of the filter posts. Scale bar is 50 μm.

FIG. 4 shows an exemplary depiction of the effects of packed spheroid size and spheroid growth. (A) Fill fraction increased as a function of the age of spheroid cultures prior to packing. (B) Packed 11-day-old spheroid. (C) Packed 18-day-old spheroid. D) Bright field images of tumor mass growing in the microfluidic device, acquired at 0, 24, and 43 hours.

FIG. 5 shows an exemplary depiction of the microenvironment gradients in tumor tissue constrained by the device. (A) Viability staining showing unstained cells at the distal end of the chamber that were visibly necrotic in transmitted light images. White arrows indicate a region of newly formed dead cells bordering the edge of the chamber. (B), (C) Apoptosis staining. Cells indicated in (B) and (C) have active caspase-3, indicating commitment to programmed cell death. (D) Cellular pH. Acidic and alkaline regions are indicated.

FIG. 6 shows an exemplary depiction of the diffusion and penetration of doxorubicin and therapeutic Salmonella bacteria. (A) Bright field image of tissue used to measure drug diffusion, 24 hours after packing. (B) Quantitative fluorescence images of doxorubicin diffusing into tumor tissue. (C) Normalized concentration profiles derived from (B). Model fits (black) were calculated using the average determined diffusion coefficient and closely fit experimental values. (D), (E) Bacterial accumulation in the device following inoculation with GFP-expressing Salmonella typhimurium. Images were acquired (D) at 28.5 after 20 hours of bacterial delivery and (E) at 45 hours after 16.5 hours of bacteria-free medium delivery. White arrows indicate a growing bacterial colony at the distal end of the chamber.

FIG. 7 schematically shows different designs for microfluidic cells. (A) and (B): Image and Schematic respectively of earlier setup. The old design employed a glass slide as the substrate. Nanoports attached on holes through the glass slide were used as connectors. (C) and (D): Schematic and Image of the new setup. A petri dish was used as the substrate, luer-lock based connectors replaced nanoports, and multiple chambers were included.

FIG. 8 shows exemplary Luer-lock Connectors (Qosina). (A) Female luer-lock to barb connector. The barb was inserted into a hole drilled into the PDMS. (B) Male luer-lock connector was attached to the female luer-lock from one end and to the tubing from the other end. The grid is square with a side of 1 cm.

FIG. 9 shows an exemplary clean hole made in the PDMS by “punch-drilling” using a piece of stainless steel tubing beveled at one end as a drill bit. Scale bar represents 1 millimeter.

FIG. 10 shows an exemplary embodiment of a device with 4 chambers, tubing and valves setup. Syringe attached to I3 was used to introduce spheroids while that attached to I2 was used for long-term perfusion. The packing outlet valves P1-P4 were selectively opened while packing to direct spheroid flow. O1 was open for long-term perfusion.

FIG. 11 shows exemplary approach for directing spheroids though the device. (A) Spheroid enters. (B) was directed to the desired chamber. (C) Next spheroid enters device and (D)-(G) is directed to the next chamber. (H) The first chamber retained the original spheroid.

FIG. 12 shows exemplary packing and growth in multiple chambers of a device. (A)-(C) 3 spheroids successfully packed in 3 different chambers of a single device. Scale bar represents is 300 μm. (D) Growth of tissue over a period of 48 hours. (E) Growth of tissue over a period of 13 hours. Scale bar represents is 300 μm.

FIG. 13 shows exemplary bacterial accumulation in tumor tissue and apoptosis induction. (A) Rapid increase in bacterial colonization from 40 to 48.5 hours corresponded to increase in apoptosis within tissue. (B) Average fluorescent intensities over entire tissue as a function of time. (C) Percentage increase in apoptosis over entire tissue. (D) The tissue was divided into 10 regions of equal width to analyze region-specific tissue response to bacterial accumulation. (E) Region-specific increase in the apoptosis after bacterial inoculation.

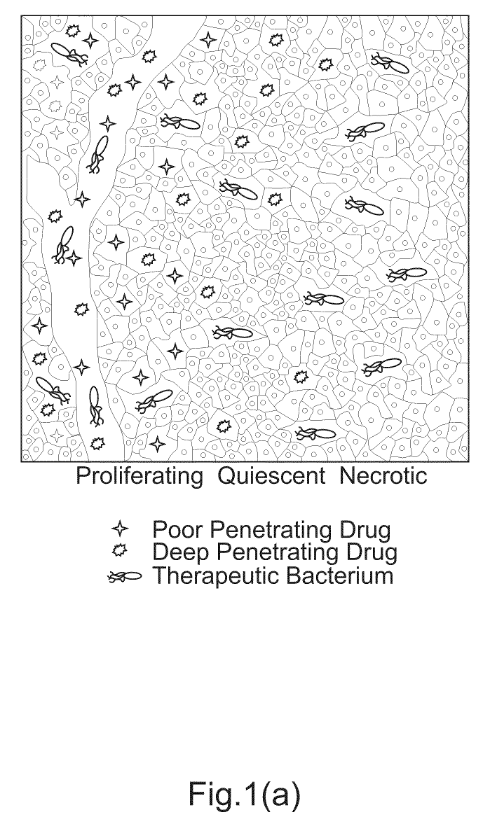

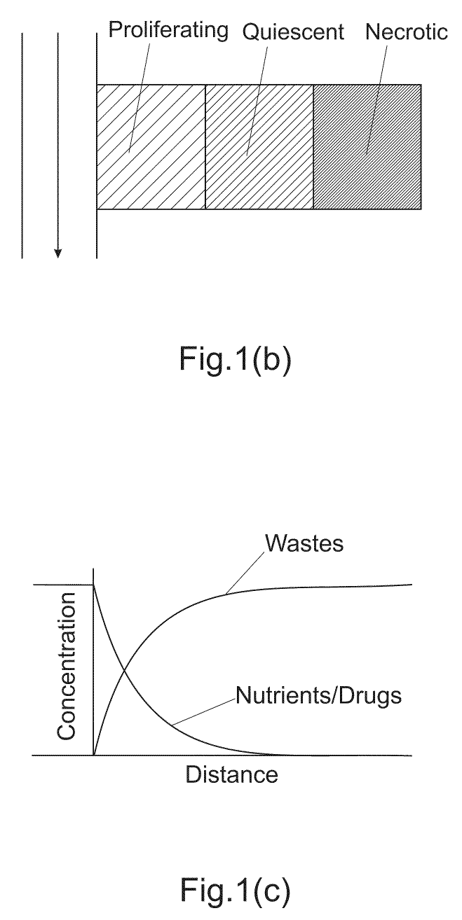

### DEFINITIONS

Unless defined otherwise, all technical and scientific terms used herein have the same meaning as is commonly understood by one of skill in the art. The following definitions are provided to help interpret the disclosure and claims of this application. In the event a definition in this section is not consistent with definitions elsewhere, the definition set forth in this section will control.

As used herein, the term “cell aggregate” refers a group of cells forming a three-dimensional space, generally resulting from forces applied along multiple axes. In certain embodiments, a “cell aggregate” refers to a tumor spheroid.

As used herein, the term “tumor” refers to a neoplasm, i.e., an abnormal growth of cells or tissue and is understood to include benign, i.e., non-cancerous growths, and malignant; i.e., cancerous growths including primary or metastatic cancerous growths. The term “neoplastic” means of or related to a neoplasm.

As used herein, the term “test agent” refers to any compound, composition or cell that can be tested as a potential therapeutic or diagnostic agent. In certain embodiments, a test agent promotes cell death of proliferating or quiescent cells or stem cells. In other embodiments, a test agent inhibits mitosis. In yet other embodiments, a test agent can target one or more signaling pathways. In other embodiments, a test agent may contribute to the apoptosis of cancer stem cells either alone or in combination with other therapeutic agents or treatment protocols such as radiation.

Examples of test agents that can be used include, but are not limited to small molecules, ligand-binding molecules such as antibodies or antibody fragments, siRNAs, shRNAs, nucleic acid molecules (RNAs, DNAs, or DNA/RNA hybrids), polynucleotides, oligonucleotides, antisense oligonucleotides, aptamers, ribozymes, peptides, peptide mimetics, amino acids, carbohydrates, lipids, organic molecules, vitamins, hormones, natural products, and the like. In certain embodiments, a test agent includes, but is not limited to, biological cells or parts of biological cells, such as microorganisms, immune cells or genetically-engineered cells. In other embodiments, a test agent can be a genetically-engineered virus, such bacteriophage or animal virus such lentiviruses or genetically engineered pseudoviruses. In some embodiments, an agent can be isolated or, in other embodiments, not isolated. As a non-limiting example, an agent can be a library of agents. If a mixture of agents is found to be a modulator, the pool can then be further purified into separate components to determine which components are in fact modulators of a target activity.

As used herein, the term “ligand-binding” refers to a member of a binding pair, i.e., two different molecules wherein one of the molecules specifically binds to the second molecule through chemical or physical means. In addition to antigen and antibody binding pair members, other binding pairs include, as examples without limitation, biotin and avidin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), and the like. Furthermore, binding pairs can include members that are analogs of the original binding member, for example, an analyte-analog or a binding member made by recombinant techniques or molecular engineering. If the binding member is an immunoreactant it can be, for example, a monoclonal or polyclonal antibody, a recombinant protein or recombinant antibody, a chimeric antibody, a mixture(s) or fragment(s) of the foregoing, as well as a preparation of such antibodies, peptides and nucleotides for which suitability for use as binding members is well known to those skilled in the art. A ligand-binding member may be a polypeptide affinity ligand (see, for example, U.S. Pat. No. 6,326,155, the contents of which are hereby incorporated by reference herein in its entirety). In one embodiment, the ligand-binding member is labeled. The label may be selected from a fluorescent label, a chemiluminescent label or a bioluminescent label, an enzyme-antibody construct or other similar suitable labels known in the art. In other embodiments, the ligand-binding molecule is conjugated to another molecule such as a toxin, e.g., ricin.

As used herein, the term “antibody” includes both polyclonal and monoclonal antibodies and may be an intact molecule, a fragment thereof (such as Fv, Fd, Fab, Fab′ and F(ab)′2 fragments, or multimers or aggregates of intact molecules and/or fragments; and may occur in nature or be produced, e.g., by immunization, synthesis or genetic engineering.

In some embodiments, antibodies suitable for the invention may include humanized or human antibodies. Humanized forms of non-human antibodies are chimeric Igs, Ig chains or fragments (such as Fv, Fab, Fab′, F(ab)′2 or other antigen-binding subsequences of Abs) that contain minimal sequence derived from non-human Ig. Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Riechmann et al, Nature 332(6162):323-7, 1988; Verhoeyen et ah, Science. 239(4847):1534-6, 1988.). Such “humanized” antibodies are chimeric Abs (U.S. Pat. No. 4,816,567, the contents of which are hereby incorporated herein in its entirety), wherein substantially less than an intact human variable domain has been substituted by the corresponding sequence from a non-human species. In some embodiments, humanized antibodies are typically human antibodies in which some CDR residues and possibly some FR residues are substituted by residues from analogous sites in rodent Abs. Humanized antibodies include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fc), typically that of a human Ig (Riechmann et al, Nature 332(6162):323-7, 1988; Verhoeyen et al. Science. 239(4847):1534-6, 1988.).

Human antibodies can also be produced using various techniques, including phage display libraries (Hoogenboom et al, MoI Immunol. (1991) 28(9): 1027-37; Marks et al, J Mol Biol (1991) 222(3):581-97) and the preparation of human monoclonal antibodies (Reisfeld and Sell, 1985, Cancer Surv. 4(1):271-90). Similarly, introducing human Ig genes into transgenic animals in which the endogenous Ig genes have been partially or completely inactivated can be exploited to synthesize human antibodies. Upon challenge, human antibody production is observed, which closely resembles that seen in humans in all respects, including gene rearrangement, assembly, and antibody repertoire (Fishwild et al., High-avidity human IgG kappa monoclonal antibodies from a novel strain of minilocus transgenic mice, Nat Biotechnol. July 1996: 14(7):845-51; Lonberg et al., Antigen-specific human antibodies from mice comprising four distinct genetic modifications, Nature Apr. 28, 1994; 368(6474):856-9; Lonberg and Huszar, Human antibodies from transgenic mice, Int. Rev. Immunol. 1995;13(1):65-93; Marks et al., By-passing inummization: building high affinity human antibodies by chain shuffling. Biotechnology (N Y). July 1992; 10(7):779-83).

As used herein, “chemotherapeutic agents,” refers to as anti-tumor or anti-cancer agents, such as cytotoxic and cytostatic agents.

### DETAILED DESCRIPTION OF THE INVENTION

The microfluidic device of the invention mimics the microenvironment gradients present in tumors and possesses many of the desired traits of the desired device: (1) enables simple introduction of cells, (2) creates predictable linear microenvironment gradients, (3) is easy to image microscopically, (4) is stable for long-term growth, and (5) can be used to test diffusion and localization of cancer therapies. The device was designed to mimic the tumor characteristics that reduce drug efficacy; a three-dimensional cell mass that limits molecular diffusion, low pH environments, and regions of therapeutically resistant cells.

In one aspect, the invention generally relates to a microfluidic device for mimicking a physiological characteristic of a three-dimensional cell mass. The microfluidic device includes a cell culture chamber for culturing a cell aggregate (the cell culture chamber having a proximal end and a distal end); a flow channel in fluid communication with the cell culture chamber at the proximal end; one or more inlets connected to the flow channel for medium in-flow or cell aggregate packing; one or more outlets connected to the flow channel for medium out-flow or removal of cell debris; and a flow system for maintaining medium flow. A filter is placed at the distal end of the cell culture chamber. The filter allows fluid flow-through while retains the cell aggregate in place within the cell culture chamber. At least a portion of the cell culture chamber is optically accessible through a transparent window.

In some embodiments, the microfluidic device may further include a temperature-controllable housing for placing the cell culture chamber therein.

The flow channel is disposed at an angle (e.g., 90 degrees) with respect to the cell culture chamber. In some preferred embodiments, the cell culture chamber comprises a planar transparent window allowing the cell aggregate to present a uniformed sample surface. A portion of the cell chamber can be optically accessible to fluorescence and time-lapse microscopy and the internal surface of the cell culture chamber can have a cell non-adhesive or cell-adhesive surface. In some embodiments, a whole side of the cell chamber is optically accessible.

The cell aggregate may be aggregates of neoplastic cells, apoptotic, quiescent and proliferating cells, cancer stem cells or tumor spheroids. The internal surface of the cell culture chamber may have a cell non-adhesive surface or a cell adhesive surface. The volume of the cell chamber depends on the applications.

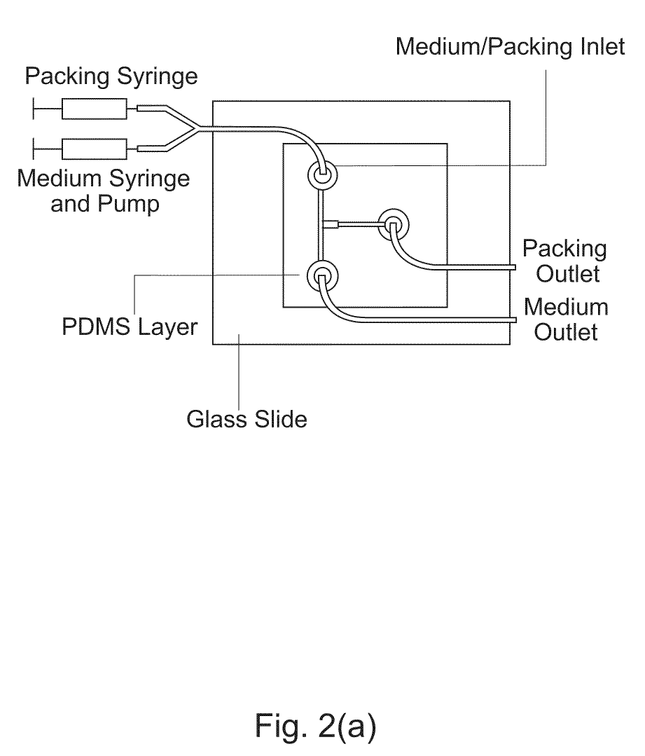

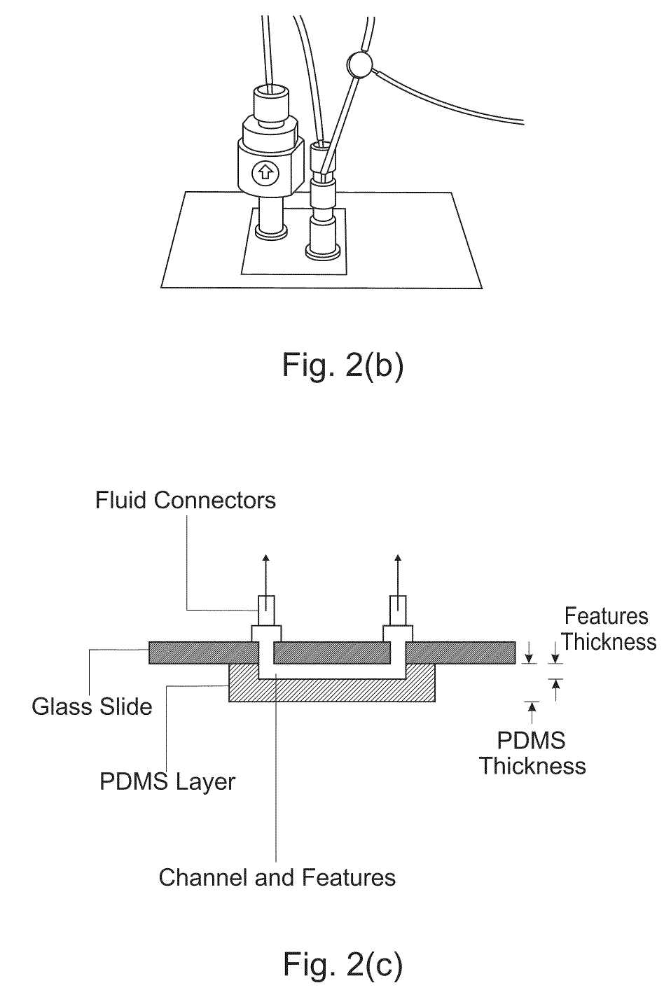

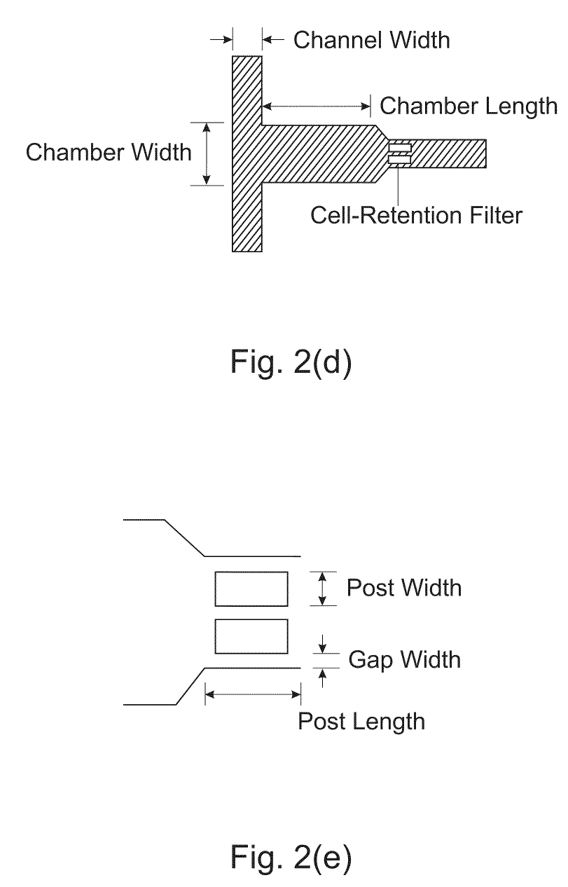

A series of devices were fabricated and tested. Each device, consisted of a cell culture chamber, a flow channel, and connections to external tubing and a syringe pump to supply a constant flow of medium (FIG. 2). In certain preferred designs, the flow channel was greater than 5000 mm long, which is greater than three times the width of each tested cell culture chamber. This length was long enough to ensure well-established laminar flow at the entrance to the cell culture chamber. A “spade”shape was used for the inlet and outlet ports to minimize volume and prevent cells from being trapped at the entrance to the flow channel.

Various designs and dimensions were tested. In all exemplary designs the total width of the filter was 250 mm, the thickness of the posts was greater than 30 mm, and the total area of open space (sum of the gap widths) was designed to be as large as possible. As the number of posts increased, the thickness decreased. The aspect ratio of the posts (width to length) was 2:1 for each design.

Design variables include: Three variable design elements were modified by changing the photolithographic mask: (1) the chamber geometry, (2) the width of the flow channel, and (3) the geometry of the filter (FIG. 2D-E). A number of different chamber geometries were created and tested (FIG. 2D). Each had a unique width (from 350 to 1500 mm) and length. Three different aspect ratios (width to length; 1:1, 1:2 and 1:4) were tested. Two different channel widths were investigated: 125 and 250 mm (FIG. 2D). Five different filter geometries were created and tested (FIG. 2E).

PDMS layers containing imprints of the microfluidic devices were created using soft lithography. Device designs were drawn using Illustrator (Adobe Systems Incorporated, San Jose, Calif.) and printed on high quality, 100 mm polyester-based Imagesetting film using an emulsion-based process (PageWorks, Cambridge, Mass.) to create micron-precision photolithographic masks. Negative images of the device features were made on photoresist-coated 100 mm silicon wafers (WaferWorld, West Palm Beach, Fla.). Wafers were coated with SU-8 2100 photoresist (Micro-chem, Newton Mass.) and spun at 1800 RPM to a thickness of 150 mm. Photoresist-coated wafers were covered with the litho-graphic masks and exposed to a 380 nm ultra-violet (UV) light source for 60 seconds to crosslink the photoresist. Non-cross-linked photoresist was removed by washing with SU-8 Developer (Microchem, Newton, Mass.) in a Pyrex dish under continual agitation. A final ethanol wash was performed to remove residual photoresist, and the wafer was allowed to air dry. A 2 mm thick PDMS (Sylgard 184, Dow Corning) layer containing an imprint of the design was cast by pouring a 10:1 mixture of monomer and curing agent over the photoresist relief. The PDMS was degassed for 20 minutes in a vacuum chamber to remove bubbles, cured for 2 hr at 60° C., and physically cut from the mold.

Completed microfluidic devices consisted of single PDMS layers adhered to a glass slide, nanoport connectors, external tubing, and a syringe pump (FIG. 2B). Holes were drilled into the glass slides using 34 mm diamond bits matching the locations of the inlet and outlet ports in the PDMS layer. The PDMS layer was adhered to the glass slide by subjecting both to oxygen plasma treatment at 200 mTorr for 7 minutes (Harrick Plasma Cleaner), assembling within 60 seconds of exposure, and heating overnight with applied pressure to improve adhesion. NanoPort connectors (Upchurch Scientific, Oak Harbor, Wash.) were attached to the glass slide directly above the holes in the slide using adhesive rings supplied by the manufacturer (Upchurch Scientific; FIG. 2C). Adhesion of the connectors was enhanced by warming for 24 hrs. A flow system, consisting of inlet and outlet flows, was connected to the fluid ports to enable continuous medium delivery (FIG. 2A-B). Cell packing required three ports and four flow streams: a packing inlet, a medium inlet, a packing outlet, and a medium outlet (FIG. 2A-B). The packing inlet and the medium inlet were joined by a Y-valve prior to the inlet port (FIG. 2A). A check valve (Upchurch Scientific) was added to the packing outlet stream to regulate the internal pressure of the device (FIG. 2B).

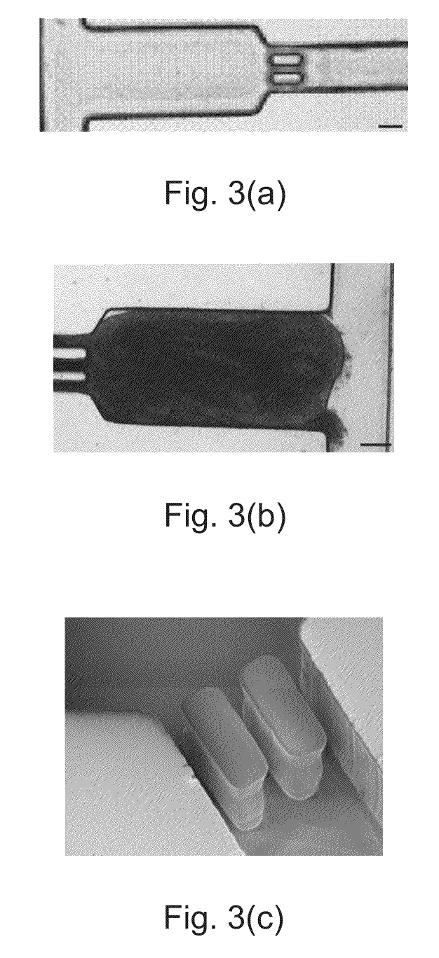

Adhesion of the polymer layer to the glass surface prevented direct insertion of spheroids into the chamber of the device. To fill cells into the chamber, spheroids were inserted through the channels and trapped by a filter at the chamber's distal end (FIG. 3A). Sealing the polymer layer to the glass slide created many advantages including, improved device sterilization, creation of well-defined borders for the cell mass, and prevention of leakage.

Five different filter geometries were designed to test the efficacy of increasing filter elements. The optimum filter contained two elements (FIG. 3A). Elements greater than 60 μm thick had uniform straight walls with minimal, overhang or curvature (FIG. 3C). As the number of elements increased, thickness decreased. All filter elements thinner than 60 μm broke dining polymer casting or attachment to the glass. This occurred because thin filter elements were tall thin planes of material (150 X˜70 X˜35 nn) without structural rigidity in the vertical direction.

The addition of a check valve at the packing outlet (FIG. 2B) improved the stability of cells within the chamber and increased the reliability of the packing process. Spheroids regularly washed out of devices fabricated without a check valve. Washout occurred at multiple times in the process: when the packing outlet stream was closed, when medium flow was initiated, and when the device was physically moved. Spheroids dislodged because the pressure in the packing outlet exceeded the pressure in the medium channel (FIG. 2A). The addition of a check valve eliminated this backpressure, increased the overall stability of the system, and enabled the entire device to be physically moved. In addition, the check valve reduced the difficulty of the packing process because spheroids remained in the chamber once the filter caught them.

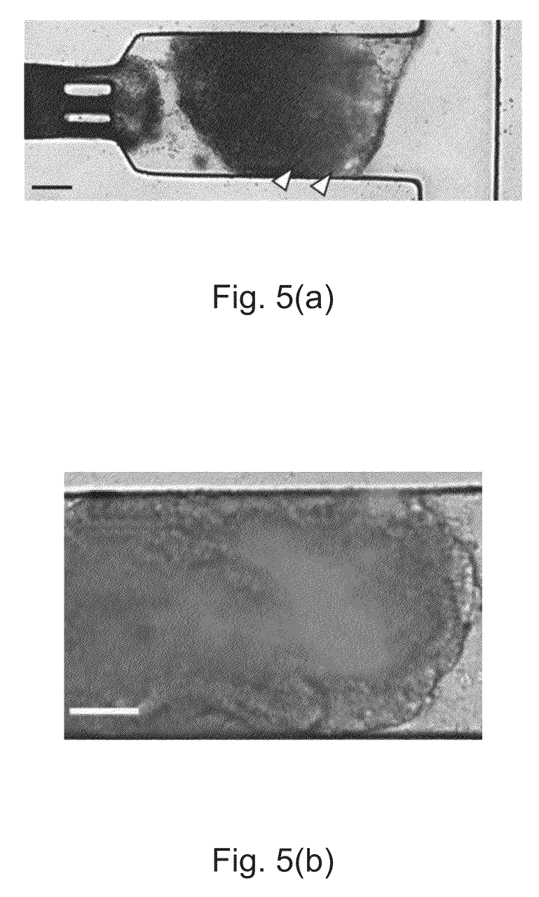

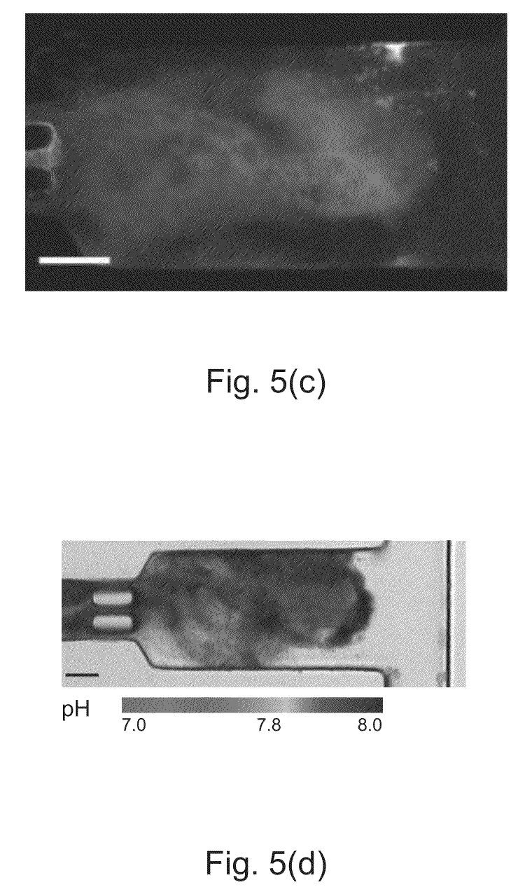

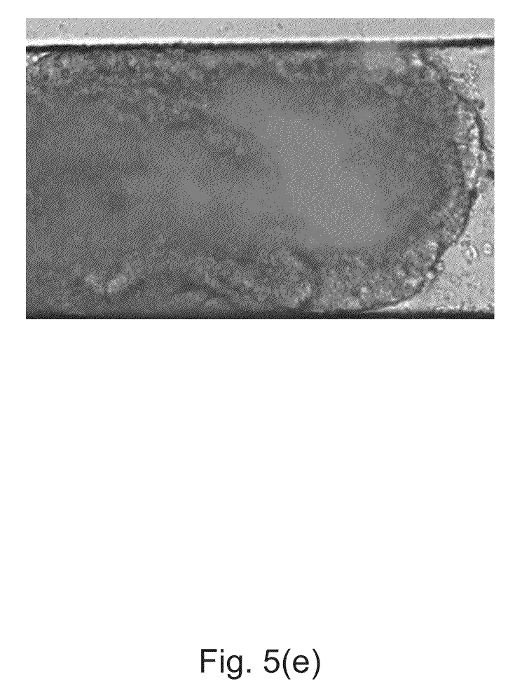

Seven different chamber geometries were tested that modulated the width and length of the cell chamber (FIG. 2D). The preferred aspect ratio was found to be 1:2. When the aspect ratio (width to length) was low (1:1), the chamber was not long enough and spheroids were washed out when medium flow was started. At the other extreme, when the aspect ratio was high (1:4), the convective medium flow did not interact with the proximal edge of the tissue mass, which grew slowly, presumably because it did not receive sufficient nutrients. Within the tested range, small chambers (350 μm wide) outperformed large chambers (500-1500 μm wide). Microenvironment gradients in both in vitro tissue and /n ⁻VIVO tumors are fully established in 100-150 μm wide chambers (see FIG. 5). Large cell masses contained a large proportion of necrotic tissue. Smaller masses were easier to form, more reproducible and more stable.

Two different channel widths were investigated: 125 and 250 μm. The larger of the two widths (250 μm) performed better because it reduced pressure gradients throughout the system. In addition, cell packing was more successful in devices with wider channels. During the packing process hand pressure was used to administer spheroid-containing medium. Devices with narrower channels had higher linear velocity and higher shear stress. As spheroids passed around the features of these devices the higher stress occasionally damaged and broke apart spheroids causing the packing process to fail.

A preferred device design adopted for further testing consisted of a T-shaped system with a 350×700×150 μm (width×length×depth) cell chamber, 250 μm channels, and three spade shaped inlet/outlet wells. A filter consisting of two posts, 65 μm wide, 130 μm long and with 40 μm gaps was located at the hack of the chamber (FIG. 2D-E). A cell-retention filter (FIG. 3) and a check valve (FIG. 2B) were used to trap tumor spheroids to till the cell chamber (FIG. 3B).

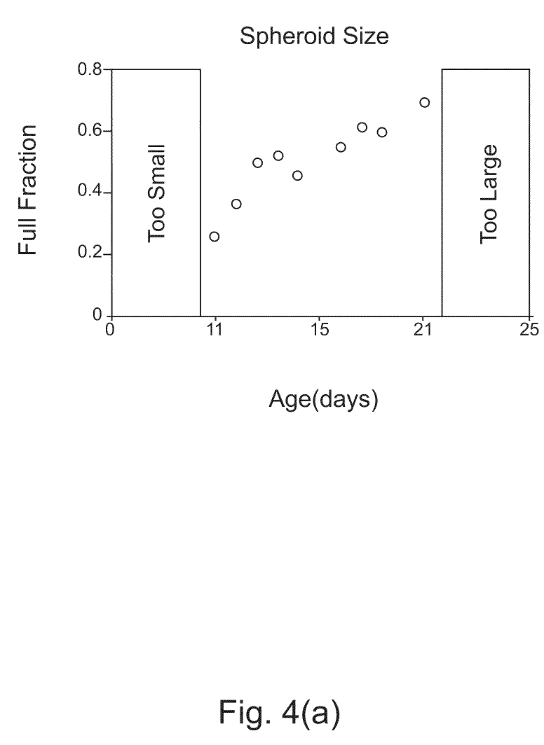

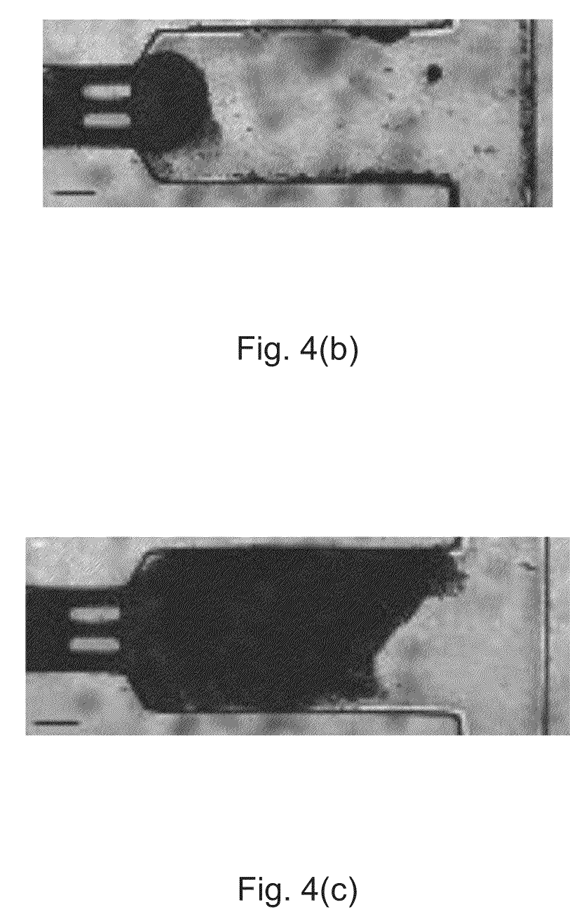

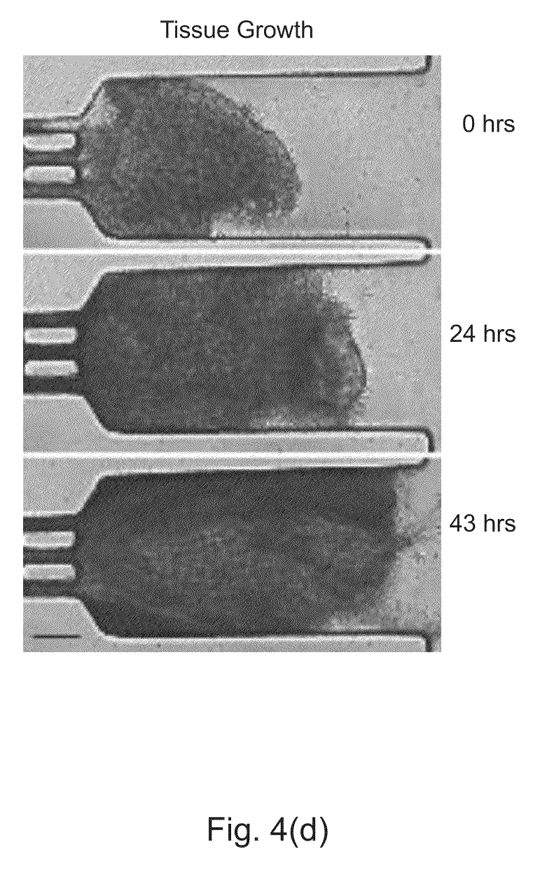

The extent that tumor tissue fills the chamber may be controlled by packing with different sized spheroids (FIG. 4A-C). Spheroids grow with increased time in culture. Spheroids less than or equal to 8 days old were too small to pack and flowed through the retention filter. Eleven-day-old spheroids successfully packed and filled approximately 25% of the chamber (FIG. 41-B). By 18 days, spheroids filled approximately 60% of the chamber (FIGS. 4A and 4C). After 21 days, spheroids became too large and fell apart during the packing process due to shear stress. The percentage chamber fill increased 3.2+/−0.1% per day of spheroid growth in culture (FIG. 4A). This dependence on age enables precise control of chamber fill for different experiments. For short-term experiments (<24 hours), 16-18 day old spheroids that fill most of the chamber should be used. For long-term experiments (>24 hours), 11-12 day old spheroids should be used to leave space for growth.

Tumor cell masses grew in the chamber at a linear rate of approximately 581 cells/hour, which corresponds to an increase in fill fraction of approximately 20% per day (FIG. 4D). At this growth rate the device is capable of performing experiments lasting up to 96 hours if small 11-day-old spheroids are used to initially pack the device. For shorter experiments, the chamber can be initially packed to 80% and allowed to equilibrate and completely fill the chamber in 24 hours. A predictable and measurable rate of growth is also useful for drug studies because it provides a comparable baseline when measuring growth reduction and cell death.

The primary functionality of the device is to create linear microenvironment gradients in in vitro tumor tissue. To test the ability of the device to produce these gradients, cell viability, the extent of apoptosis, and pH were measured as a function of position (FIG. 5).

Diffusion of fluorescent dyes was quantified using an Olympus IX71 inverted microscope with a 10× Plan-APO fluo-rescence objective and IPLab imaging software (BD Biosciences, Rockville, Md.). To create high-resolution images of the entire cell chamber, two 665.8 μm×873.9 μm fields of view were tiled together using a specialized IPLab script. Time-lapse microscopy was performed by capturing images at regular intervals using an automated stage and image acquisition script. Microenvironment gradients were defined relative to the flow channel; the end of the chamber closest to the channel was designated “proximal” and the opposite end was designated “distal.” All staining used small-molecule dyes.

Cell viability was quantified using the Live/Dead Viability/Cytotoxicity Assay Kit (Invitrogen; Carlsbad, Calif.). This assay uses calcein AM and ethidiumhomodimer (Ethd-1) to identify viable and non-viable cells, respectively. Calcein AM fluoresces proportionally to intracellular esterase activity, and Ethd-1 binds to DNA in cells with permeable membranes. To simultaneously stain both viable and non-viable cells 1:2000 (v/v) calcein AM and 1:500 (v/v) Ethd-1 in DMEM were flowed through the device at 3.5 μL/min and 25° C. Images were acquired 12 hours after packing the chamber and addition of the dyes.

The extent of apoptosis in the chamber was quantified using the CaspGLOW Red Caspase-3 Staining Kit (BioVision, Inc., Mountain View, Calif.). This assay uses DEVD-FMK conjugated to sulfo-rhodamine. DEVD-FMK is an inhibitor that irreversibly binds to activated caspase-3. The conjugated molecule (Red-DEVD-FMK) is a fluorescent marker that stains cells committed to programmed cell death. Apoptotic cells were identified by adding 1:1000 (v/v) Red-DEVD-FMK in DMEM to the 10 mL syringe and running the device at 3.5 μL/min and 25° C. Images were acquired 17 hours after packing the chamber and adding the Red-DEVD-FMK dye.

Local cellular pH was quantified using 20,70-bis-(2-carboxy-ethyl)-5-(and-6-)-carboxyfluorescein (BCECF) free acid fluorescent indicator (Invitrogen, Carlsbad, Calif.). Ratiometric measurement of BCECF fluorescence allowed concentration independent conversion from fluorescence to pH. Before staining, the device was packed and the cell mass was allowed to grow in the device at 37° C. with DMEM flowing at 3 μL/min for 24 hours. To stain for pH, 25 μM BCECF free acid in DMEM was run through the device at 3 μL/min at 37° C. After 7 hours, fluorescent images at 440 nm and 495 nm were taken after washing the device and chamber with dye-free DMEM. The fluorescence ratio was adjusted for background fluorescence and converted to pH. The maximum and minimum values of the fluorescence ratio were determined by titrating BCECF in DMEM with NaOH and HCl, respectively.

Cells were found viable at the proximal end of the cell mass next to the medium flow and were dead at the distal end (FIG. 5A). Cell death in cell masses was primarily caused by apoptosis (FIG. 5B-C). Fluorescence in FIG. 5B-C indicates the presence of activated caspase-3. which is a down-stream protease in the programmed cell-death pathway that indicates commitment to apoptosis. Staining for cellular pH indicated that the environment was acidic in the interior of the cell masses and progressively more alkaline towards the exterior (FIG. 5D). The microenvironment gradients in the tumor masses developed after cells were packed into the device. This change indicates that the geometry of the chamber limited the availability of nutrients to predictably and controllably create proliferating and dead regions. The spheroids used to pack the device contained regions of viable cells and necrosis arranged in a radial pattern. If this pattern was maintained after packing, a region of viable cells would be visible along the edges of the tissue masses bordering the chamber walls (FIG. 5A, indicated with arrows). However, the cells along the edge of the chamber were not viable; indicating the shape of the chamber and limited diffusion of nutrients rearranged the radial gradients creating a linear microenvironment pattern in less than 12 hours.

The microfluidic device may be designed to provide multiple cell culture chambers allowing simultaneous measurement of multiple tissues samples. In another aspect, the invention generally relates to a microfluidic device for mimicking a physiological characteristic of a plurality of three-dimentional cell masses. The microfluidic device includes a plurality of cell culture chambers for culturing a plurality of cell aggregates. Each cell culture chamber has a proximal end and a distal end. The microfluidic device further includes one or more flow channels in fluid communication with each cell culture chamber at the proximal end one or more inlets connected to each flow channel for medium in-flow or cell aggregate packing; one or more outlets connected to each flow channel for medium out-flow or removal of cell debris; and a flow system for maintaining medium flow in the one or more flow channels. A filter is placed at the distal end of each cell culture chamber and allows fluid flow-through while retaining the cell aggregate in place within the cell culture chamber. At least a portion of the cell culture chamber is optically accessible through a transparent window.

The microfluidic devices of the invention successfully mimic the microenvironment gradients present in tumors (FIG. 5) and can be used for long-term experiments (FIG. 4). Using this experimental platform, the efficacy of therapeutic agents may be tested on a wide variety of neoplastic cells from benign or malignant tumors of different origins.

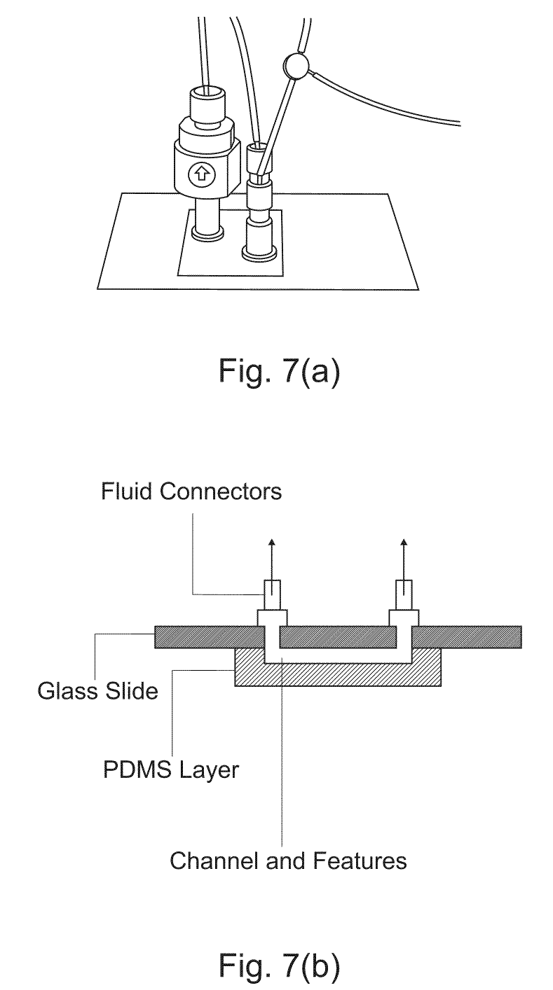

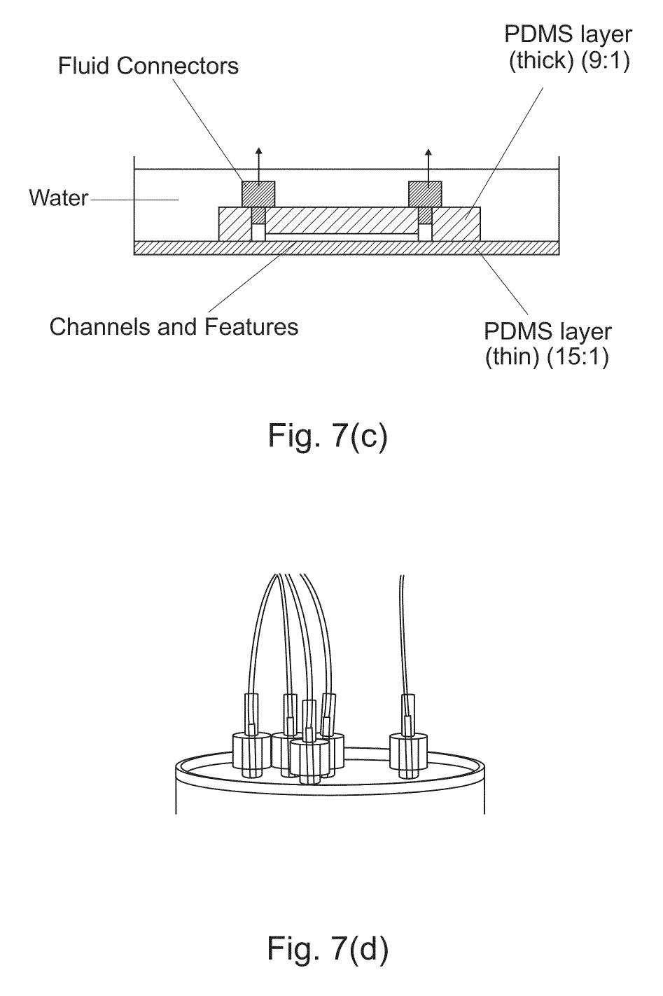

Several modifications may be made to the above designs to achieve: (1) Simplified fabrication; (2) Improved performance, and (3) Increased throughput. For example, (1) Glass slides may be replaced by plastic petri dishes. These provide, a higher working area so that more features can be incorporated in a single device. Further, petri dishes provide space to immerse the entire device assembly under water, avoiding evaporation from within the device (FIG. 7C, D). (2) Plasma treatment may be used to bond substrate to PDMS has been replaced by the “ratio-mismatch” bonding technique. This technique is simpler and makes stronger bonds. (3) Luer-lock based barbed connectors instead of nanoports, inserted directly into the PDMS. These connectors are significantly cheaper, allow decreasing dead volumes at the device-tubing connection, and are transparent allowing imaging at the point of entry into the device. (4) Rather than check valves (Upchurch Scientific), which had a large space footprint and were expensive, inexpensive shut-off valves may be installed off-chip. This helps conserve real estate on device. Furthermore, multiple chambers may be incorporated into the device allowing higher throughput testing of drugs/therapeutics.

Neoplastic cells here refers to cells characteristic of any cellular-proliferative disease state of any organ, including but not limited to:

Hematologic disease states: blood (myeloid leukemia (acute and chronic), acute lymphoblastic leukemia, chronic lymphocytic leukemia, hairy cell leukemia, myeloproliferative diseases, multiple myeloma, myelodysplastic syndrome), Hodgkin's disease, non-Hodgkin's lymphoma (malignant lymphoma):

Genitourinary tract disease states: kidney (adenocarcinoma, Wilms tumor (nephroblastoma), lymphoma, leukemia, renal cell carcinoma, renal pelvis carcinoma, nephroma, teratoma, sarcoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Cardiac disease states: sarcoma (angiosarcoma, fibrosarcoma, rhabdomyosarcoma, liposarcoma), myxoma, rhabdomyoma, fibroma, lipoma and teratoma;

Nervous system disease states: brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma), skull (osteoma, hemangioma, granuloma, xanthoma, osteitisdeformians), meninges (meningioma, meningiosarcoma, gliomatosis);

Lung disease states: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatoushanlartoma, mesothelioma, sarcoidosis;

Gastrointestinal disease states: small bowel (adenocarcinoma, lymphoma, carcinoid tumors. Karposi's sarcoma, leiomyoma, hemangioma, lipoma, neurofibroma, fibroma), large bowel (adenocarcinoma, tubular adenoma, villous adenoma, hamartoma, leiomyoma); esophagus (squamous cell carcinoma, adenocarcinoma, leiomyosarcoma, lymphoma), stomach (carcinoma, lymphoma, leiomyosarcoma), pancreas (ductaladenocarcinoma, insulinoma, glucagonoma, gastrinoma, carcinoid tumors, vipoma);

Liver disease states: hepatoma (hepatocellular carcinoma), cholangiocarcinoma, hepatoblastoma, angiosarcoma, hepatocellular adenoma, hemangioma;

Bone disease states: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginousexostoses), benign chondroma, chondroblastoma, chondromyxofibroma, osteoidosteoma and giant cell tumors;

Gynecological disease states: uterus (endometrial carcinoma), cervix (cervical carcinoma, pre-tumor cervical dysplasia), ovaries (ovarian carcinoma [serous cystadenocarcinoma, mucinouscystadenocarcinoma, unclassified carcinoma], granulosa-thecal cell tumors, Sertol/Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonalrhabdomyosarcoma), fallopian tubes (carcinoma), breast; and

Skin disease states: malignant melanoma, basal cell carcinoma, squamous cell carcinoma, Kaiposi's sarcoma, moles dysplastic nevi, lipoma, angioma, dermatofibroma, keloids or psoriasis.

In certain embodiments, the neoplastic cells to be used in the microfluidic device may be immortalized cancer cell lines. In other embodiments, the cells may be derived directly from human tumors. Where a test agent is effective at inducing apoptosis of quiescent cells within a human in vitro tumor, the same untreated tumors may be transplanted into humanized mice to determine if the test agent is equally effective on tumors grown in vivo.

In another aspect, the invention generally relates to a method for mimicking a physiological characteristic of a three-dimensional cell mass. The method includes: providing a cell culture chamber for holding a cell aggregate, the cell culture chamber having a proximal end and a distal end; providing a flow channel in fluid communication with the cell culture chamber at the proximal end; seedling a cell aggregate in the cell culture chamber, wherein a filter at the distal end of the cell culture chamber retains the cell aggregate in the cell culture chamber; and culturing the cell aggregate in the cell culture chamber, wherein culture medium flowing through the flow channel can diffuse into cell culture chamber. The method can mimic a characteristic of the microenvironments surrounding blood vessels in a tissue, such as a tumor tissue. The method may further include measuring a transport or targeting property of an agent in a tumor tissue. In certain embodiments, the method may further include a radiation step.

In yet another aspect, the invention generally relates to a method for screening a compound for a therapeutic property. The method includes: providing a cell culture chamber for holding a cell aggregate, the cell culture chamber having a proximal end and a distal end; providing a flow channel in fluid communication with the cell culture chamber at the proximal end; culturing the cell aggregate in the cell culture chamber, wherein a filter at the distal end of the cell culture chamber retains the cell aggregate in the cell culture chamber; adding a test agent to the culture medium flowing through the flow channel; allowing diffusion of the test agent from the flow channel to the cell culture chamber; and recording a change in a therapeutic property of the cell aggregate in response to the presence of the test agent. In certain embodiments, the method may further include a radiation step.

Because the design of the device is simple, it may be employed to create a plurality of in vitro tumors for rapid drug development and tuning. The cell-retention filter at the rear of the cell culture chamber enables automated spheroid insertion for high-throughput robotic drug screening. Many therapeutics that have promise in monolayer cell screens are ineffective in solid tumors. The present invention provides a rapid and efficient platform for testing novel test agents or novel combinations thereof.

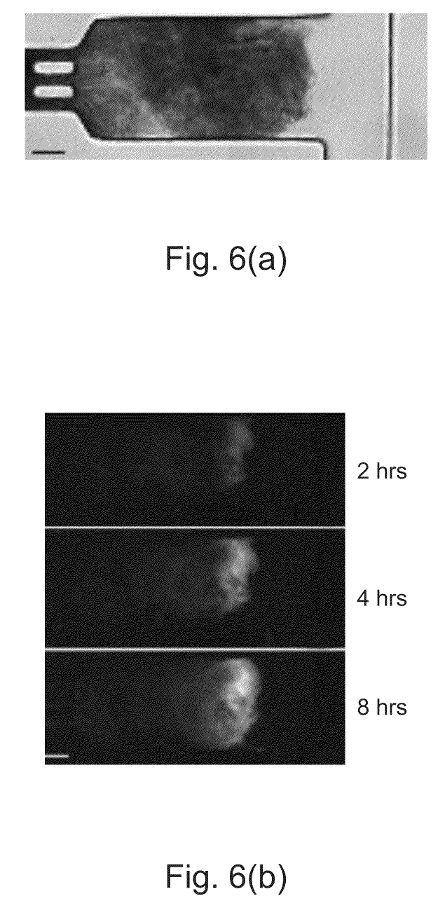

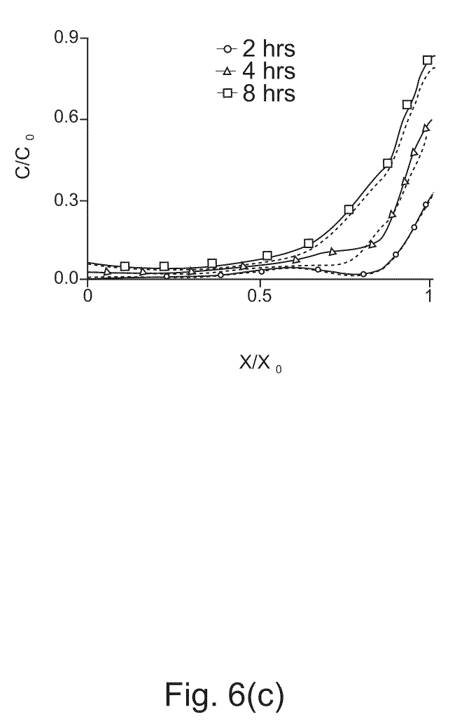

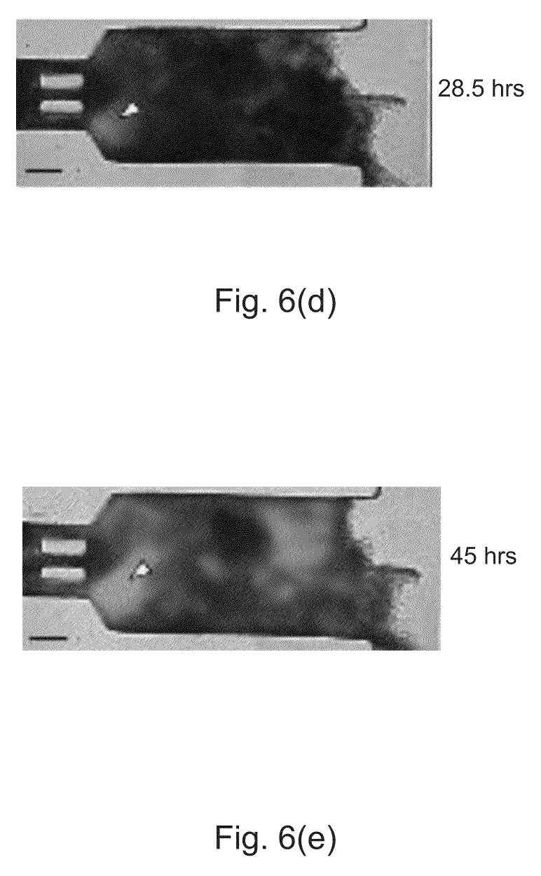

The ability to model drug diffusion through a tumor (FIG. 6) allows prediction of drug concentrations at any position in the tumor at any time based on a drug concentration in the blood. This information can be used to determine appropriate dosages and predict the effectiveness of novel cancer therapies based on their ability to diffuse to different regions of a tumor. The potential to perform long-term experiments enables drug delivery protocols that mimic physiological pharmacokinetics. In addition, the ability to observe and measure microenvironment changes in both time and space (FIG. 5) enables accurate predictions of drug efficacy. Many cancer drugs, e.g. paclitaxel, function by inducing apoptosis in tumors. By comparing to an established baseline (FIG. 5B), the extent that drug increases apoptosis and its location can be quantified. In certain embodiments, an agent can sensitize tumor cells to the activity of a second agent

Another attribute of the microfluidic device of the invention is the ability to identify different cellular compartments of a tumor and monitor the effect of test agents on the different cell compartments in real time.

Cancer stein cells (CSCs) are cancer cells (found within tumors or hematological cancers) that possess characteristics associated with normal stem cells, specifically the ability to give rise to all cell types found in a particular cancer sample. CSCs are therefore tumorigenic (tumor-forming), in contrast to other non-tumorigenic cancer cells. CSCs may generate tumors through the stem cell processes of self-renewal and differentiation into multiple cell types. Such cells are proposed to persist in tumors as a distinct population of quiescent cells that may cause relapse and metastasis by giving rise to new tumors.

The experimental platform described herein provides a unique opportunity to study the genesis and evolution of CSCs within tumors. For example, CSCs may be identified in situ by transforming the cells with recombinant constructs comprising a cancer stem-cell specific promoter driving the expression of a reporter molecule such as a GFP, green fluorescent protein. Cell transformation can be achieved either by transfection of cultured cells with the genetic constructs or by transduction of the cells with a pseudovirus such as a lentivirus that is genetically engineered to carry the reporter construct. In one embodiment, the CSC promoter may be the Oct-4 or nanog promoter.

Other examples of stem cell-specific promoters that may be used are disclosed in U.S. Pat. No. 7,396,680. Other methods of identifying and isolating cancer stem cells are taught in U.S. Pat. No. 7,217,568.

Alternatively, for any particular tissue-specific tumor type, a library of pseudovirus each containing a different promoter—reporter combination can be transduced into cancer cells to determine which promoters are active within the CSC compartment.

In other embodiments, agents may be tested for their ability to mobilize the CSC compartment therebymaking them susceptible to more conventional cytotoxic or cytostatic chemotherapeutics. The efficacy of test agent on tumor cells and CSCs in particular can be further evaluated by measuring whole genome gene expression either by RNA or protein microarray analysis.

The microfluidic device also provides a platform to test the efficacy of tumor-infiltrating lymphocytes or dendritic cells on tumors generated in vitro.

### Experimental

### Administration and Chemotherapeutic Diffusion of Doxorubicin

The rate of diffusion of doxorubicin (Dox), a common chemotherapeutic agent, was measured in the microfluidic device using time-lapse fluorescence imaging. The concentration of Dox was measured directly because it naturally fluoresces at 515 nm after excitation with 480 nm light. Medium containing 10 μM Dox was delivered to the device at 3 μL/min for 32 hours at 25° C. Diffusion of Dox was quantified using an Olympus IX71 inverted microscope with a 10× Plan-APO fluorescence objective and IPLab imaging software (BD Biosciences, Rockville, Md.). Fluorescence images were taken every 30 minutes. The images were adjusted so that each time point displayed the same pixel value range and intensity values. Linear fluorescence intensity profiles were created by averaging the intensity of all pixels at a given linear distance from the front of the cell mass using a macro created in ImageJ (NM Research Services Branch). Fluorescence intensity profiles were converted to concentration profiles by subtracting the background fluorescence and multi-plying by the known concentration (10 mM) in the channel.

The effective diffusion coefficient (D) was calculated by modeling Dox transport through the system as simple Fickian diffusion through a semi-infinite solid.

$$\frac{\partial {C}_{Dox}}{\partial t} = D \frac{{\partial }^{2} {C}_{Dox}}{\partial {x}^{2}} \qquad ( 1 )$$

Boundary conditions were established by assuming that initially (t 1/4 0) there was no Dox in the cell mass; the Dox concentration at the distal end of the chamber (x 1/4 N) was zero; and the Dox concentration at the proximal end (x 1/4 0) equaled the concentration in the channel.

$${{C}_{Dox} \vert}_{t = 0} = 0 {{C}_{Dox} \vert}_{x = ∞} = 0 {{C}_{Dox} \vert}_{x = 0} = {C}_{Dox , channel} \qquad ( 2 )$$

Modeling diffusion using Equation (1) assumes 1) that the concentrations in the channel are constant and well mixed; 2) that the cell mass is square, 3) that edge effects are minimal, and 4) that diffusion can be described with an effective diffusion coefficient. Least squares error analysis and the Solver function in Excel were used to fit the error function solution (Equation 3) to the concentration profile at 2, 4, 6, and 8 hours.

$$\frac{{C}_{Dox , channel} - {C}_{Dox} ( x , t )}{{C}_{Dox , channel}} = erf ( \frac{x}{2 \sqrt{Dt}} ) \qquad ( 3 )$$

To calculate the best-fitting diffusion coefficient for each concentration profile at a one time, the left side of Equation 3 was calculated at all positions. Then a diffusion coefficient was guessed, the right side of Equation 3 was calculated, and the residual error between the two sides was determined. The guessed diffusion coefficient was adjusted to minimize the sum of the squares of all residuals. An average diffusion coefficient was determined from the values calculated at each of the four time points.

As shown in FIGS. 6A and 6B, the diffusion of doxorubicin in tumor masses in the device was quantified by acquiring a time-lapse series of fluorescence and bright field images as described above. The Dox concentration in the cell masses increased as a function of time and depth (FIG. 6B). By fitting a diffusion model to linear fluorescence intensity profiles (FIG. 6C), the diffusion coefficient was calculated to be 8.75×10⁷ cm²/s, which agrees with the previously reported value of 9.1×10⁷ cm²/s in human breast cancer. The closeness of the fits shows that diffusion can be effectively modeled using the device. One limitation of this technique to measure diffusivity is that the drug molecule must be naturally fluorescent and adding a fluorescent tag may considerably affect the transport properties. However, this methodology has an advantage over standard protocols, such as multicellular layer culture, because drug concentration can be measured continuously as a function of time and position.

### Administration and Penetration of Anti-Cancer Bacteria

A single colony of green-fluorescent-protein-expressing Salmonella typhimurium was suspended in LB with 250 kanamycin and shaken for 4 hours at 37° C. This bacterial culture was centrifuged to remove the supernatant and re-suspended in cell culture medium (DMEM) with 250 μg/ml kanamycin to an estimated concentration of 1×10⁶ CFU/ml. The device was packed and allowed to equilibrate for 8.5 hours at 37° C. as described above. Medium flow was kept at 3 μl/min throughout. An initial background image was acquired, and a syringe filled with the bacteria-containing medium was attached to the feed line. The medium syringe and pump were maintained at 4° C., to limit bacterial growth. Transmitted and fluorescent pictures were taken at 28.5 hours, and a syringe containing medium without bacteria was attached to the feed line. At 45 hours final transmitted and fluorescent images were taken.

After twenty hours of flowing bacterial medium, fluorescent bacteria had penetrated to the distal end of chamber where they grew to concentrations considerably greater than in the channel (FIG. 6D). Wild-type Salmonella are known to chemotax towards necrotic tumor tissue and preferentially grow there. After flowing bacteria-free medium for 16.5 hours, the bacterial density continued to increase in the necrotic distal end of the cell masses (FIG. 6E, arrow).

### Cell and Spheroid Culture

Human LS174T colon carcinoma cells were maintained in Dulbecco's Modified Eagles Medium (DMEM: Sigma Aldrich, St. Louis, Mo.) containing 10% fetal bovine serum (FBS) at 37° C. and 5% CO₂. Tumor spheroids were formed by inoculating a single-cell suspension into culture flasks coated with poly(2-hydroxyethylmethacrylate), which prevented cell adhesion to the flask surface.

### Cell Packing by Filter Retention

Introducing cells after the PDMS layer was adhered to the glass required that cells be “packed” into the chamber. Packing was achieved by flowing spheroids through the tubing and trapping them in a filter at the distal end of the chamber (FIG. 2D-E). Prior to inserting cells, the assembled bioreactor was sterilized by flushing with 70% ethanol, followed by PBS to remove air and residual ethanol. A 10 mL syringe was filled with medium, connected to the inlet stream, and placed in a syringe pump (Harvard Apparatus; FIG. 2N). Between five and ten spheroids were added to medium in a 1 mL syringe, which was connected to the packing inlet stream (FIG. 2A). The medium outlet was crimped closed, and the cell solution was injected through the reactor until a spheroid was trapped by the filter. Once the chamber was filled, the packing outlet was closed, the medium flow outlet was opened, and the syringe pump was started. A nominal flowrate of 3.0 mL/min produced an average linear velocity similar to that of blood in tumors (1.7 mm/sec). The entire device was maintained at 37° C. in an enclosed environment that surrounded the microscope stage.

### Spheroid Size

The effect of packing spheroids of different size was investigated by using different aged spheroids. Spheroid size can be controlled by varying the growth time in culture. The fill fraction was defined as the ratio of the area of the packed cell mass to the total area of the chamber. The area of the cell mass was determined from transmitted light images and visual inspection. Fill fractions represent averages of multiple measurements from each day. For each packing, the ability for spheroids to be retained by the filter and the quality of the cell mass was noted.

### Long Term Growth

Tumor growth in the device was measured using time-lapse microscopy. A small spheroid was packed to fill approximately 50% of the chamber and allowed to grow. Medium was run through the device at 3 μL/min for 43 hours, and the device was incubated at 37° C. Transmitted light images were taken at 0, 24, and 43 hours. The number of cells in the chamber was calculated by dividing the volume of packed tissue mass by the average cell volume, which was assumed to be 524 μm³, based on a uniform 5 μm radius. The rate of growth was determined by linear regression.

### PDMS-Substrate Bonding

An alternative way to bond PDMS chips to glass substrates was by the “ratio-mismatch” bonding technique that involved bringing 2 surfaces of PDMS with different concentrations of curing agent—one partially cured while the other fully cured—in contact with each other. A 100 mm diameter plastic culture dish (Fisher Scientific) replaced the previously used glass slides as the substrate. 10 g of PDMS was mixed with 0.66 g of the curing agent (15:1 PDMS: curing agent) and poured onto the substrate forming a thin layer over it. After removal of bubbles by degassing, this layer was cured at 60° C. for 45-60 minutes forming a partially cured sticky polymer layer. A fully cured thick layer of PDMS (9:1 PDMS: curing agent) with the flow channels and features on the bottom surface was brought in contact with this partially cured layer, and the system was further baked at (50° C. overnight. This allowed a strong bond to form in between the substrate and the thin PDMS layer and more importantly in between the two PDMS layers. As compared to oxygen plasma treatment, this technique was simpler, formed stronger bonds and was more reliable.

### Tubing to Device Connectors

Several techniques of attaching tubing to the microfluidic system were explored, including:
a) Inserting tubing directly into the PDMS while it cured,
b) Inserting blunt syringe needles (Integrated Dispensing Solutions, Part #9991279-2) into holes in the PDMS.
c) Using external-to-device cubes of PDMS as connectors, and
d) Using luer-lock connectors with barbs.

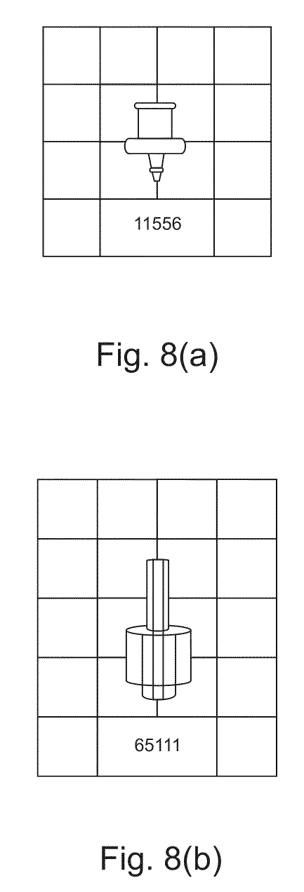

Luer-lock connectors with barbs, inserted directly into holes in the PDMS, were found to provide the preferred solution. Female luer-lock to barb connectors (Qosina, Part #11556) (FIG. 8A) were inserted into the PDMS chips, and connected to male luer-lock connectors (Qosina, Part #65111) (FIG. 8B) into which the tubing was inserted. The size of the hole in the PDMS was critical and was chosen to be slightly smaller in diameter than the diameter of the barb on the connector to ensure a tight seal. This technique was simple and effective. Furthermore, since holes were made directly on the PDMS with the features, the sensitive step involving alignment of substrate and PDMS was eliminated.

### “Punch-Drilling”

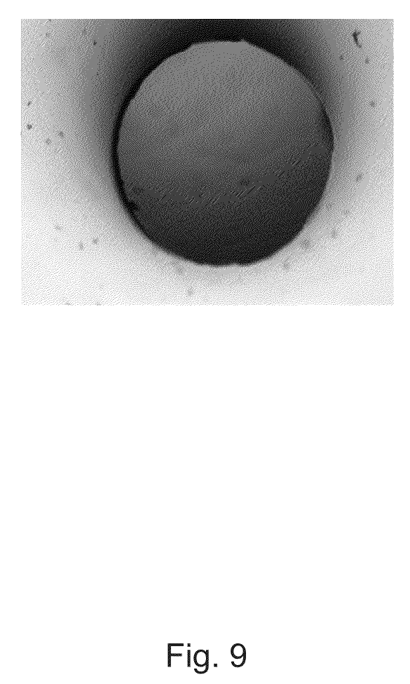

Making holes in the PDMS can pose challenges. Drilling may end up giving a rough surface and plenty of debris, which is particularly undesirable since they could lead to obstruction of spheroid flow. Punching, on the other hand, has a tendency to tear the PDMS and sometimes gives unclean holes. Punching worked better on thinner PDMS Chips, but using the bier-lock barb connector demanded the PDMS be at least 7.5 mm thick to allow the entire barb to be accommodated within the hole. A combination of the two techniques, i.e., “Punch-Drilling”, was utilized to create clean holes in the thick PDMS layer. A punch was mounted on a drill press and used as a hollow drill bit. The punch itself was a piece of thin walled stainless steel tubing (McMaster-Can) cut into three-quarter inch pieces. The tips of the pieces were beveled using sand paper. Such beveled pieces of steel tubing were used as hollow drill bits for punch-drilling. The preferred PDMS to curing agent ratio for these holes to be clean was found to be 9:1. Clean holes were obtained even within very thick PDMS layers by this method (FIG. 9).

### Device Throughput

Current techniques for testing for efficacy of drugs can only test drug efficacy on monolayers of cells that do not aptly reproduce the heterogeneities that exist in tumors. The microfluidic device of the invention may be used to provide a better platform for drug testing—especially cancer therapeutics—as it recreates the heterogeneities in-vitro.

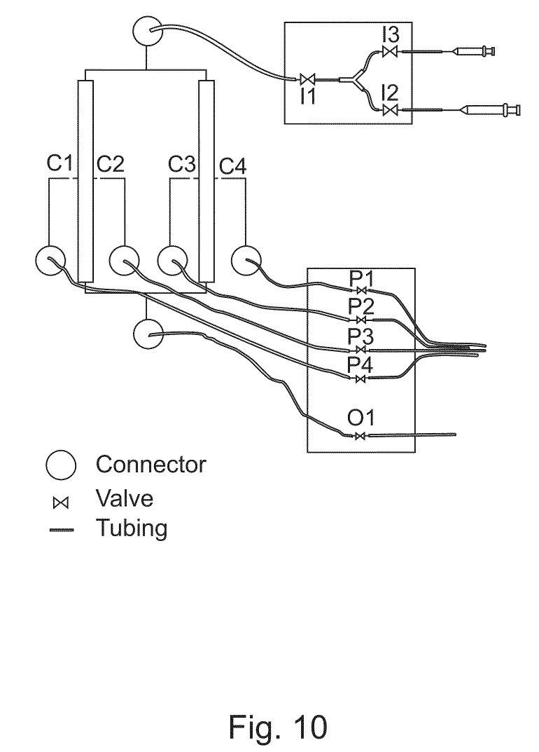

A device containing 8 chambers for simultaneously testing 8 tumor tissues for 2 different treatments (4 for each treatment) was designed fabricated and tested. FIG. 10 shows the schematic of the device with 4 chambers; the actual device included 2 sets of such features.

The device consisted of a main inlet channel that split into 4 channels, distributing the flows equally within them. Chambers for tissue were built on each channel and outlet channels emerged from the back of each chamber (the packing outlets). The main flow channels, downstream the chambers, merged back into a main outlet channel. Manual shut-off valves were installed on each packing outlet (valves P1-P4) and the main outlet (valve O1). An assembly Of 3 valves (valves I1-I3) together with a Y-connector was used at the inlet. These allowed selection of inlet flows in between 2 inlet syringes—one used to introduce spheroids, while the other as the main medium inlet for long-term perfusion.

After flushing all channels with ethanol, PBS and cell culture medium in that order, spheroids were introduced into the system through the packing inlet syringe through valve I3 (while I2 remained shut). While packing into a particular chamber, the packing outlet valve corresponding to that chamber was left open, while all other outlet valves were closed. This allowed the spheroid to flow to the desired chamber, where the posts at the back of the chamber held it in place.

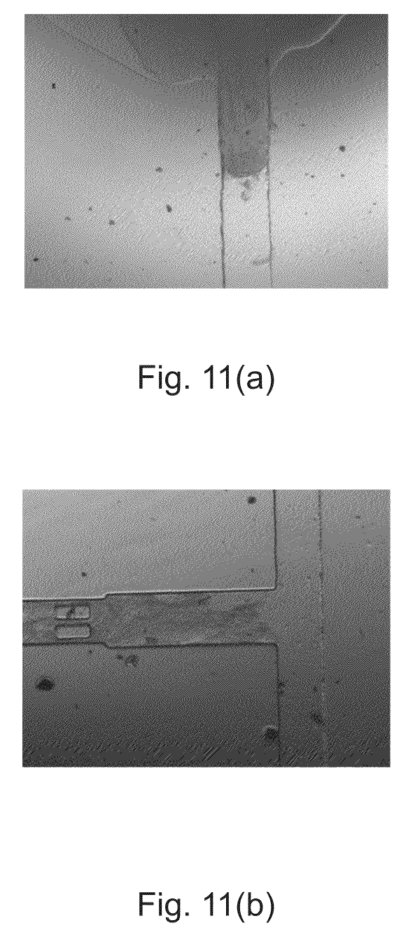

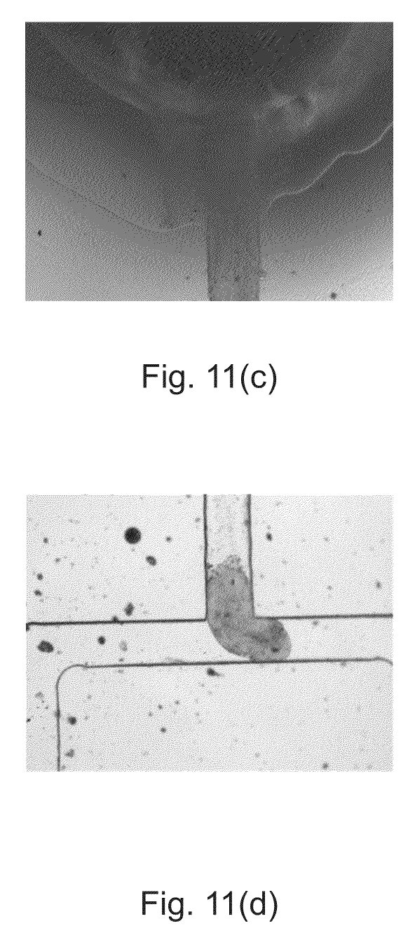

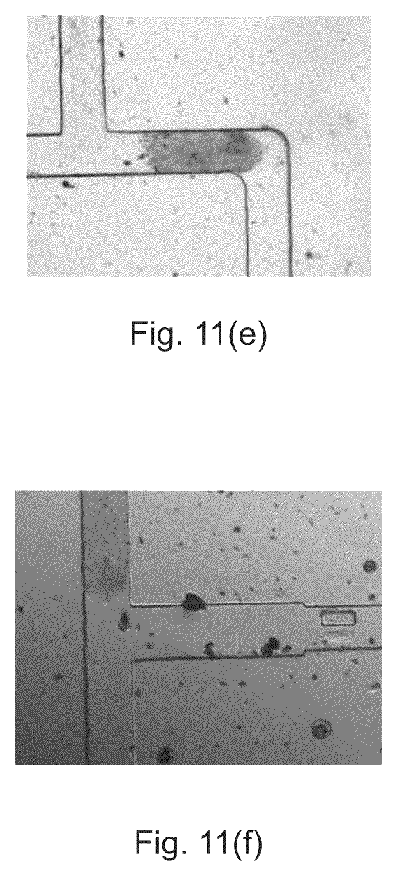

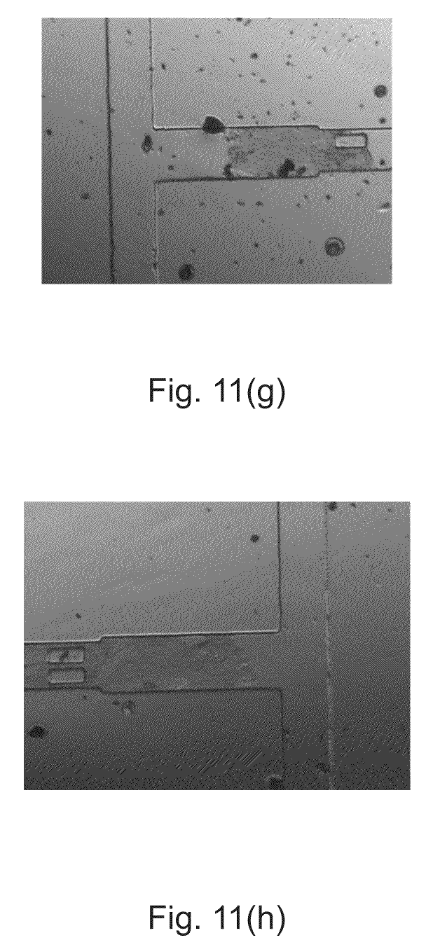

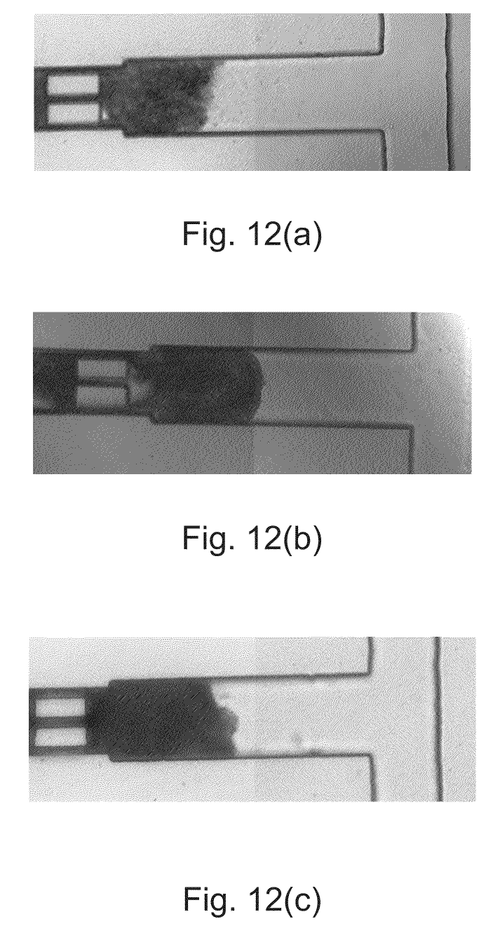

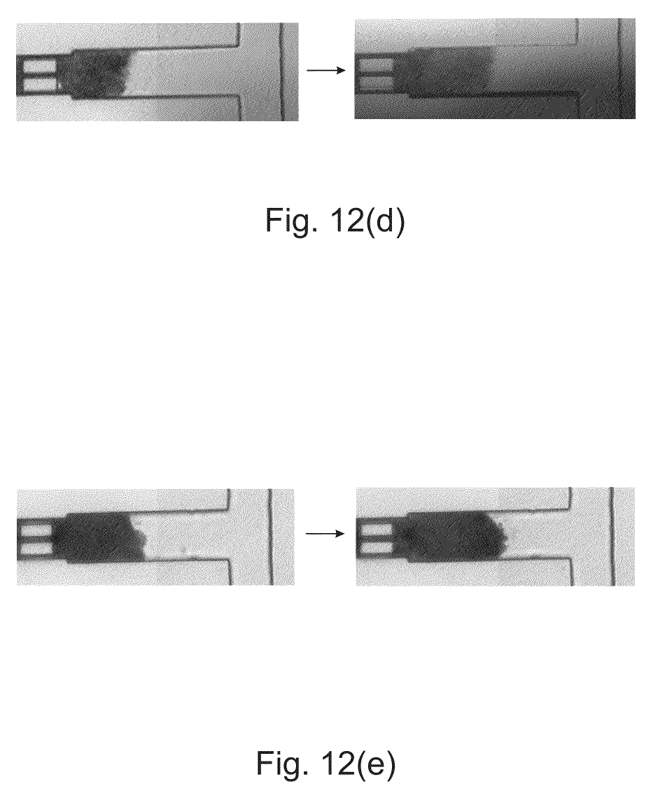

Once packed, the packing outlet valve for that chamber was shut off, and the packing outlet for the next chamber was opened. Spheroids were introduced again through the inlet syringe and directed to the desired chamber. After packing chambers, all packing outlet valves (P1-P4) were shut, while the main outlet valve O1 was opened allowing fluid to flow past all chambers. Inlet valve I1 was shut off at this moment and I2 was opened through which medium for long-term cultures, fluorescent dyes, and the required therapeutics were introduced. The path of spheroids as they were introduced into the device and directed to desired chambers was tracked (FIG. 11). A spheroid entered the device (FIG. 11A) and was directed into one of the chambers (FIG. 11B). Once in place, another spheroid was introduced which entered (FIG. 11C) and was tracked all the way up to the desired chamber (FIG. 11D-G). After packing the second chamber, the spheroid in the first chamber was still found to be in place (FIG. 11H). In another experiment 3 chambers were packed with spheroids (FIG. 12A-C). 2 out of the 3 spheroids exhibited long-term growth within the device (FIG. 12D-E).

### Testing Bacteria as Cancer Therapeutics

One of the major limitations of current cancer chemotherapeutics is ineffective penetration within the highly heterogeneous tumor tissue. Active, as opposed to passive transport of the therapeutic agent may overcome this limitation. Engineered bacteria possess the ability to actively transport deep into the tumor tissue, and can be genetically manipulated to target desired regions within the tumor and deliver therapeutic payloads. The rapid growth rate of bacteria in currently available batch systems of in vitro cultures makes it impossible to study the effect of therapeutic bacteria on mammalian tissue over physiologically relevant time scales. The microfluidic device developed here provides a good platform for performing long-term mechanistic studies on bacterial tumor therapies.

Spheroids were packed into the microfluidic device and grown for 15-18 hours to allow equilibration and development of linear nutrient gradients. A fluorescent dye that stained active caspase-3 was introduced into the system at this point and was used to quantify the induced apoptosis. After 5-8 hours of dye penetration into the tissue, a 1-hour long plug of a constitutively GFP expressing strain of Salmonella Typhimurium bacteria SL1344 (at 100,000 CFU/ml) was introduced into the system. The corresponding controls were allowed to grow without bacterial inoculation. Time-lapse images were acquired for fluorescence from bacteria as well as the fluorescent dye for up to 40 hours after inoculation.

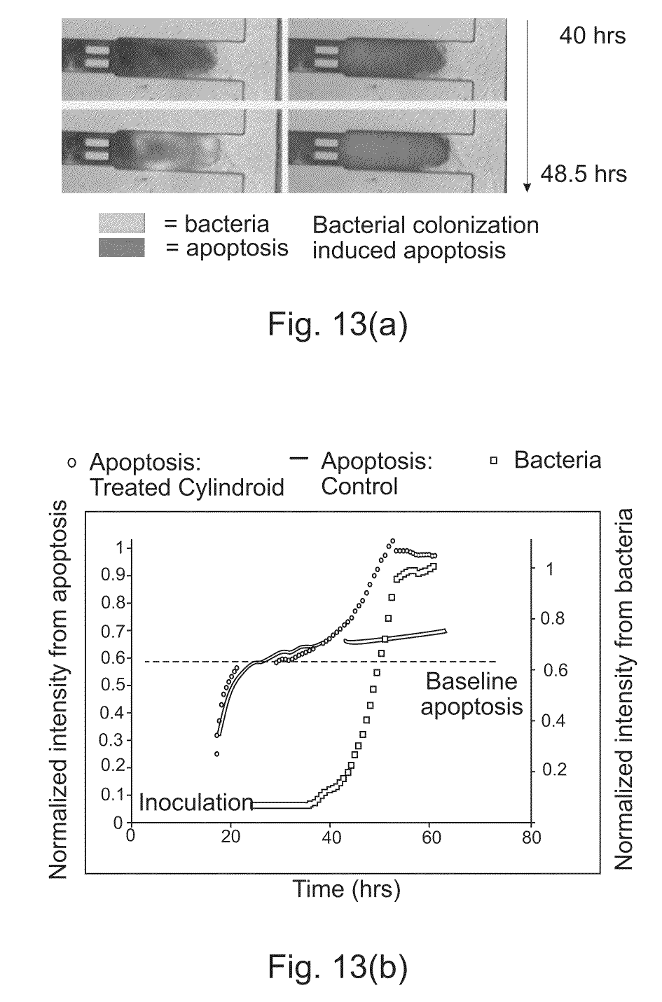

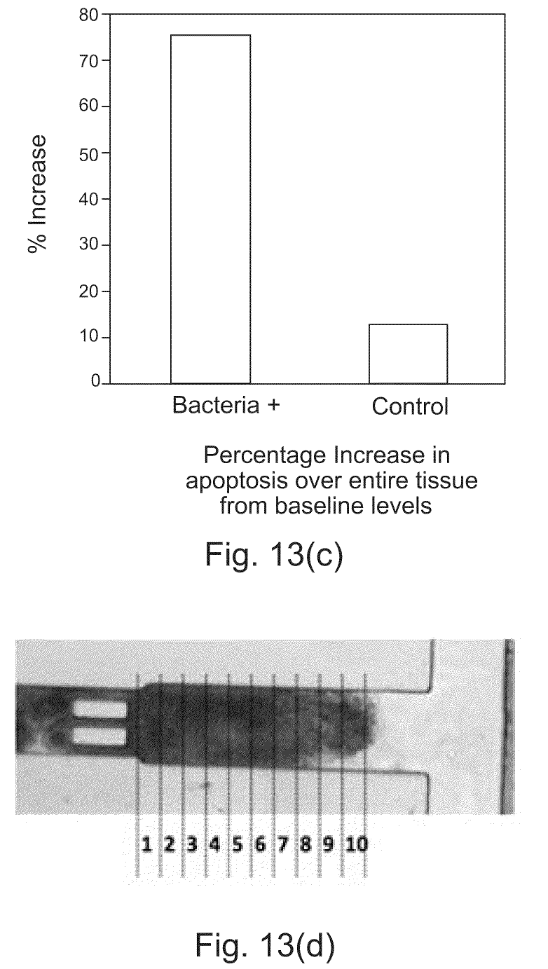

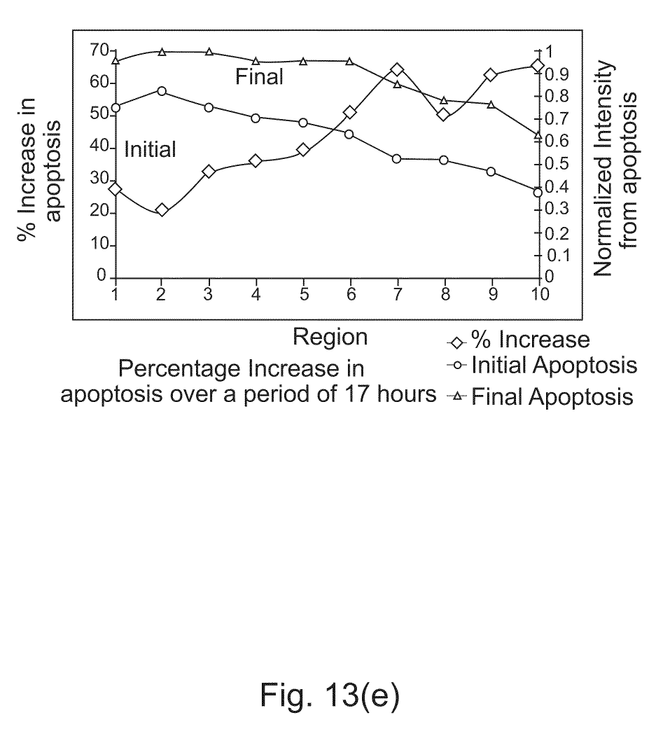

Bacteria formed colonies within the tumor tissue that were detectable by fluorescence measurements, about 15 hours after inoculation. Colonies were formed preferentially within the tumor tissue as opposed to the flow channels in experiments lasting longer than 40 hours (FIG. 13A). Average fluorescence intensities over the entire tissue were evaluated—both for bacterial fluorescence as well as that from the apoptosis dye—and plotted as a function of time (FIG. 13B). A steep increase in the concentration of bacteria was observed in between 40-50 hours. This increase in bacterial concentration corresponded to a notable increase in the extent of apoptosis induced. The corresponding control represented by tumor tissue that was not inoculated with bacteria, did not exhibit such a steep rise (FIG. 13B). The bacteria treated tissue was found to have a 75% increase in the extent of apoptosis as compared to the baseline level, while the corresponding number for untreated tissue was 15% (FIG. 13C). Further insights could be gained into the mechanism of bacterial action by quantifying apoptosis induced by bacteria as a function of location within the tissue. The tissue was divided into 10 regions of equal width and the relative increase in apoptosis over 17 hours after inoculation of bacteria was evaluated (FIG. 13D-E).

### Incorporation by Reference

References and citations to other documents, such as patents, patent applications, patent publications, journals books, papers, web contents, have been made in this disclosure. All such documents are hereby incorporated herein by reference in their entirety for all purposes,

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance which can be adapted to the practice of this invention in its various embodiments and equivalents thereof.

What is claimed is:

1. A microfluidic device for mimicking a physiological characteristic of a three-dimensional cell mass comprising:
a cell culture chamber for culturing a cell aggregate, the cell culture chamber having a proximal end and a distal end;
a flow channel in fluid communication with the cell culture chamber at the proximal end;
one or more inlets connected to the flow channel for medium in-flow or cell aggregate packing;
one or more outlets connected to the flow channel for medium out-flow or removal of cell debris; and
a flow system for maintaining medium flow,
wherein a filter is placed at the distal end of the cell culture chamber allowing fluid flow-through while retaining the cell aggregate in place within the cell culture chamber, and wherein at least a portion of the cell culture chamber is optically accessible through a transparent window.

2. The microfluidic device of claim 1, further comprising a temperature-controllable housing for placing the cell culture chamber therein.

3. The microfluidic device of claim 1, wherein the flow channel is disposed at an angle with respect to the cell culture chamber.

4. The microfluidic device of claim 3, wherein the angle is 90 degrees.

5. The microfluidic device of claim 1, wherein the cell culture chamber comprises a planar transparent window allowing the cell aggregate to present a uniformed sample surface.

6. The microfluidic device of claim 1, wherein the cell aggregate comprises cancer cells.

7. The microfluidic device of claim 1, wherein the cell aggregate comprises apoptotic, quiescent and proliferating cells.

8. The microfluidic device of claim 1, wherein the cell aggregate comprises tumor spheroids.

9. The microfluidic device of claim 1, wherein a portion of the cell culture chamber is optically accessible to fluorescence and time-lapse microscopy.

10. The microfluidic device of claim 1, wherein the internal surface of the cell culture chamber comprises a cell non-adhesive surface.

11. The microfluidic device of claim 1, wherein the internal surface of the cell culture chamber comprises a cell adhesive surface.

12. A microfluidic device for mimicking a physiological characteristic of three-dimentional cell masses comprising:
a plurality of cell culture chambers for culturing a plurality of cell aggregates, each cell culture chamber having a proximal end and a distal end;
one or more flow channels in fluid communication with each cell culture chamber at the proximal end;
one or more inlets connected to each flow channel for medium in-flow or cell aggregate packing;
one or more outlets connected to each flow channel for medium out-flow or removal of cell debris; and
a flow system for maintaining medium flow in the one or more flow channels,
wherein a filter is placed at the distal end of each cell culture chamber allowing fluid flow-through while retaining the cell aggregate in place within the cell culture chamber, and wherein at least a portion of the cell culture chamber is optically accessible through a transparent window.

13. The microfluidic device of claim 12, further comprising one or more temperature-controllable housings for placing the plurality of cell culture chambers therein.

14. The microfluidic device of claim 12, wherein each flow channel is disposed at an angle with respect to each cell culture chamber.

15. The microfluidic device of claim 12, wherein each cell aggregate comprises cancer cells.

16. The microfluidic device of claim 12, wherein each cell aggregate comprises apoptotic, quiescent and proliferating cells.

17. The microfluidic device of claim 12, wherein each cell aggregate comprises tumor spheroids.

18. The microfluidic device of claim 12, wherein a portion of each cell culture chamber is optically accessible to fluorescence and time-lapse microscopy.

19. A method for mimicking a physiological characteristic of a three-dimensional cell mass, the method comprising:
providing a cell culture chamber for holding a cell aggregate, the cell culture chamber having a proximal end and a distal end;
providing a flow channel in fluid communication with the cell culture chamber at the proximal end;
seeding a cell aggregate in the cell culture chamber, wherein a filter at the distal end of the cell culture chamber retains the cell aggregate in the cell culture chamber; and
culturing the cell aggregate in the cell culture chamber, wherein culture medium flowing through the flow channel can diffuse into cell culture chamber.

20-32. (canceled)

33. A method for screening a compound for a therapeutic property, comprising:
providing a cell culture chamber for holding a cell aggregate, the cell culture chamber having a proximal end and a distal end;
providing a flow channel in fluid communication with the cell culture chamber at the proximal end;
culturing the cell aggregate in the cell culture chamber, wherein a filter at the distal end of the cell culture chamber retains the cell aggregate in the cell culture chamber;
adding an test agent to the culture medium flowing through the flow channel;
allowing diffusion of the test agent from the flow channel to the cell culture chamber; and
recording a change in a therapeutic property of the cell aggregate in response to the presence of the test agent.

34-53. (canceled)
